(19) United States
(12) Patent Application Publication
(10) Pub. No.: US 2014/0088069 A1
(43) Pub. Date: Mar. 27, 2014

(54) NOVEL ANTIBACTERIAL COMBINATION THERAPY

(71) Applicants: Eric D. Brown, Oakville (CA); Maya Farha, Ancaster (CA); Alexander Leung, Richmond Hill (CA); Gerry Wright, Cambridge (CA); Kalinka Koteva, Hamilton (CA); Ted Sewell, Ottawa (CA); Linda Ejim, Hamilton (CA)

(72) Inventors: Eric D. Brown, Oakville (CA); Maya Farha, Ancaster (CA); Alexander Leung, Richmond Hill (CA); Gerry Wright, Cambridge (CA); Kalinka Koteva, Hamilton (CA); Ted Sewell, Ottawa (CA); Linda Ejim, Hamilton (CA)

(21) Appl. No.: 14/093,307

(22) Filed: Nov. 29, 2013

Related U.S. Application Data
(63) Continuation-in-part of application No. PCT/CA2012/000533, filed on Jun. 1, 2012.
(60) Provisional application No. 61/492,098, filed on Jun. 1, 2011.
(60) Provisional application No. 61/624,266, filed on Apr. 14, 2012.

(51) Int. Cl. C07D 217/04; A61K 31/4365; A61K 31/545; A61K 31/47; A61K 31/43; A61K 31/431; A61K 31/546; A61K 31/472

(52) U.S. Cl. CPC C07D 217/04; A61K 31/546; A61K 31/4365; A61K 31/472; A61K 31/47; A61K 31/43; A61K 31/431; A61K 31/545
USPC 514/196; 514/207; 546/149; 546/147; 514/198; 514/199; 514/202; 514/203; 514/204

(57) ABSTRACT

An antibacterial composition is provided including a combination of a β-lactam antibiotic that has a binding affinity for bacterial penicillin-binding protein 2; and a non-antibiotic compound which may be a thienopyridine or a non-thienopyridine compound. A method of treatment using the composition is also provided.

### FIELD OF THE INVENTION

The present invention relates to the treatment of bacterial infections and, in particular, to therapies that include a combination of a non-antibiotic drug and a β-lactam antibiotic.

### BACKGROUND OF THE INVENTION

Over the past 20 years, there has been an explosion in the prevalence of antibiotic resistant bacterial infections, both in the hospital and in the general community. Notably, methicillin-resistant Staphylococcus aureus (MRSA) has emerged as one of the most important human pathogens. In fact, treatment of MRSA infection in patients often involves the only remaining antibiotics that are active against the strain such as vancomycin, or linezolid, even though β-lactams were once the most celebrated antibiotics.

The problem of resistance is compounded by the decreasing rate of discovery of novel antibiotics, creating a pressing need for new strategies for treatment. One approach is through the combination of known drugs; specifically looking for non-antibiotic drugs that can potentiate the activity of existing antibiotics. There is a growing understanding that drugs with proven therapeutic activity for a particular use often have uncharacterized potential for alternate use. In addition, previously approved drugs have well characterized toxicology and pharmacology properties, offering significant drug development advantages.

There is, thus, a need to develop alternative antibacterial treatments that are effective against one or more antibiotic-resistant bacterial strains.

### SUMMARY OF THE INVENTION

Non-antibiotic drugs have now been identified which are effective to potentiate the activity of β-lactam antibiotics to treat bacterial infection, and in particular, to treat antibiotic-resistant bacterial infections.

Accordingly, in one aspect of the invention, a novel composition is provided comprising a β-lactam antibiotic in combination with a non-antibiotic compound having the following general formula (I):

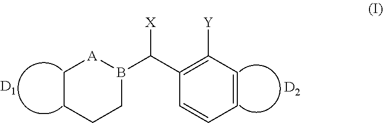

wherein:
A may be C or N, and B may be C or B may be N when A is C;
X is selected from the group consisting of H, OH, CH₂OH, CH₂F, CHF₂, CF₃, C₁-C₆ alkyl, —COOH, —COR, —COOR, NO₂, NH₃, NH₂R, NHR₂, wherein R is selected from cyclic, linear or branched C₁-C₆ alkyl and may be the same or different in NHR₂ ;
Y is selected from the group consisting of H, OH, halogen (e.g. Br, Cl, F and I), C₁-C₆ alkyl, NO₂, NH₃, NH₂R¹, NH(R¹)₂, CF₃, CH₂OH, CH₂F, CHF₂, wherein R¹ is selected from halogen and linear or branched C₁-C₆ alkyl and may be the same or different in NH(R¹)₂;
D₁ may be phenyl, substituted or unsubstituted thiophene, substituted or unsubstituted furan or substituted or unsubstituted pyrrole, wherein the substituents are selected from OH, halogen (e.g. Br, Cl, F and I), cyclic, linear or branched C₁-C₆ alkyl and OR², wherein R² is selected from C₁-C₆ alkyl and —COR; and
D₂ is either phenyl or nil.

In another aspect of the invention, a method of treating a bacterial infection in a mammal is provided. The method comprises administering to the mammal an effective amount of a β-lactam antibiotic and a non-antibacterial compound having the general formula (I).

In another aspect, an article of manufacture is provided. The article of manufacture comprises packaging material containing a composition. The composition comprises a β-lactam antibiotic and a non-antibacterial compound having the general formula (I). The packaging material is labeled to indicate that the composition is useful to treat a bacterial infection in a mammal.

In another aspect of the present invention, use of a β-lactam antibiotic and a non-antibacterial compound having the general formula (I) is provided to treat a bacterial infection in a mammal.

In a further aspect, use of a β-lactam antibiotic and a non-antibacterial compound having the general formula (I) is provided for the manufacture of a medicament for the treatment of a bacterial infection in a mammal.

These and other aspects of the invention will be described by reference to the following figures.

### BRIEF DESCRIPTION OF THE DRAWINGS

FIG. 1 depicts results of a high-throughput screen of previously approved non-antibiotic drugs for synergism with cefuroxime (A) and the strategy used in narrowing down selected compounds (B); and

FIG. 2 is a sample dose variation (checkerboard) analysis of ticlopidine in combination with cefuroxime illustrating the synergistic interaction between the drugs.

### DETAILED DESCRIPTION OF THE INVENTION

The present invention provides an anti-bacterial composition comprising a β-lactam antibiotic in combination with a non-antibiotic compound. The composition is useful to treat bacterial infection including Staphylococcus aureus strains, such methicillin-resistant Staphylococcus aureus (MRSA) and methicillin-sensitive Staphylococcus aureus (MSSA).

β-lactam antibiotics that are useful in the present composition include those that have a binding affinity for bacterial penicillin-binding protein 2 (PBP2). Examples of suitable β-lactam antibiotic include, but are not limited to, Ampicillin, Cefaclor, Cefotaxime, Ceftizoxime, Cefoxitin, Cefuroxime, Cephalothin, Cloxacillin, Nafcillin, Oxacillin, Penicillin G, Piperacillin and pharmaceutically acceptable salts, solvates or prodrugs of any of these antibiotics. These antibiotics are commercially available,

The composition also comprises a non-antibiotic compound having the following general formula (I):

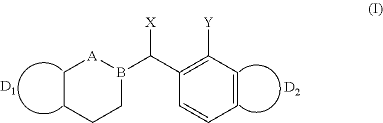

wherein:
A may be C or N, and B may be C or B may be N when A is C;
X is selected from the group consisting of H, OH, CH₂OH, CH₂F, CHF₂, CF₃, C₁-C₆ alkyl, —COOH, —COR, —COOR, NO₂, NH₃, NH₂R, NHR₂, wherein R is selected from cyclic, linear or branched C₁-C₆ alkyl and may be the same or different in NHR₂;
Y is selected from the group consisting of H, OH, halogen (e.g. Br, Cl, F and I), C₁-C₆ alkyl, NO₂, NH₃, NH₂R¹, NH(R¹)₂, CF₃, CH₂OH, CH₂F, CHF₂, wherein R¹ is selected from halogen and linear or branched C₁-C₆ alkyl and may be the same or different in NH(R¹)₂;
D₁ may be phenyl, substituted or unsubstituted thiophene, substituted or unsubstituted furan or substituted or unsubstituted pyrrole, wherein the substituents are selected from OH, halogen (e.g. Br, Cl, F and I), cyclic, linear or branched C₁-C₆ alkyl and OR², wherein R² is selected from C₁-C₆ alkyl and —COR; and
D₂ is either phenyl or nil.

For clarity, C₁-C₆ alkyl includes linear and branched alkyl groups. Examples of suitable alkyl groups include, but are not limited to, methyl, ethyl, propyl, isopropyl, butyl, isobutyl, sec-butyl, tert-butyl, pentyl, isopentyl, 3-methylpentyl, hexyl and isohexyl. Cyclic C₁-C₆ alkyl includes, but is not limited to, cyclopropyl, cyclobutyl and cyclopentyl.

In one embodiment, the non-antibiotic compound is a thienopyridine compound having the general formula (II):

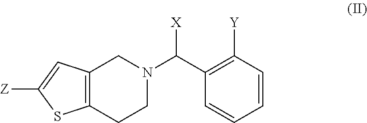

wherein:
X and Y are as defined above; and
Z is selected from H, OH, cyclic, linear or branched C₁-C₆ alkyl and OR², wherein R² is as defined above.

Examples of suitable thienopyridine compounds for use in the present composition include, but are not limited to, ticlopidine, prasugrel ((RS)-5-[2-cyclopropyl-1-(2-fluorophenyl)-2-oxoethyl]-4,5,6,7-tetrahydrothieno[3,2-c]pyridin-2-yl acetate) and clopidogrel ((+)-(S)-methyl 2-(2-chlorophenyl)-2-(6,7-dihydrothieno[3,2-c]pyridin-5(4H)-yl)acetate. Such thienopyridine compounds may be purchased, or chemically synthesized using well-established synthetic techniques.

In another embodiment, the non-antibiotic compound is a non-thienopyridine compound having the general formula (III):

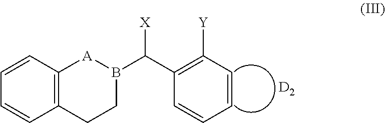

wherein:
A, B, X, Y and D₂ are as defined above.

Examples of suitable non-thienopyridine analogues for use in the present composition include, but are not limited to, 2-(2-Chloro-benzyl)-1,2,3,4-tetrahydro-isoquinoline, 2-Benzyl-1,2,3,4-tetrahydro-isoquinoline, 1-(2-Chloro-benzyl)-1,2,3,4-tetrahydro-quinoline 2-(2-Methyl-benzyl)-1,2,3,4-tetrahydro-isoquinoline, 2-(2-Fluoro-benzyl)-1,2,3,4-tetrahydro-isoquinoline, 2-(2-Nitro-benzyl)-1,2,3,4-tetrahydro-isoquinoline, 2-(2-Trifluoromethyl-benzyl)-1,2,3,4-tetrahydro-isoquinoline, (2-Chloro phenyl)-(3,4-dihydro-1H-isoquinolin-2-yl)-acetic acid methyl ester and 2-Naphthalen-2-ylmethyl-1,2,3,4-tetrahydro-isoquinoline. Such non-thienopyridine compounds may be purchased, or chemically synthesized using well-established synthetic techniques.

As one of skill in the art will appreciate, the β-lactam antibiotic or non-antibiotic compound may be utilized in the form of a pharmaceutically acceptable salt, hydrate or solvate. The term “pharmaceutically acceptable” refers to a salt, hydrate or solvate that retains the desired biological activity of the parent compound and does not impart any undesired toxicological effects (see e.g., Berge, S. M. et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. A “solvate” is formed by admixture of the β-lactam antibiotic or non-antibiotic compound in a solvent which is preferably pharmaceutically acceptable. A “hydrate” is formed by combination of the compound with water.

The β-lactam antibiotic or non-antibiotic compound may also be utilized in the form of a pharmaceutically acceptable prodrug. The term “prodrug” refers to a compound (e.g. a drug precursor) that is transformed in vivo to yield the active compound, e.g. the active β-lactam antibiotic or non-antibiotic compound as defined above. The transformation may occur by various mechanisms (e.g., by metabolic or chemical processes), such as, for example, through hydrolysis in blood.

The composition is prepared by combining an amount of each of the β-lactam antibiotic and the non-antibiotic compound to yield anti-bacterial activity, and particularly, anti-bacterial activity against the target bacteria.

The present composition may be additionally include one or more pharmaceutically acceptable adjuvants or carriers. The expression “pharmaceutically acceptable” means acceptable for use in the pharmaceutical arts, i.e. not being unacceptably toxic, or otherwise unsuitable for administration to a mammal. Examples of pharmaceutically acceptable adjuvants include, but are not limited to, diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule, lozenge, solution or suspension in an aqueous or non-aqueous liquid, an oil-in-water or water-in-oil liquid emulsion, an elixir or syrup are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, disintegrating agents, anti-oxidants, preservatives, colouring agents and flavouring agents may also be present. In another embodiment, the composition may be formulated for application topically as a cream, lotion or ointment. For such topical application, the composition may include an appropriate base such as a triglyceride base. Such creams, lotions and ointments may also contain a surface-active agent and other cosmetic additives such as skin softeners and the like as well as fragrance. Aerosol formulations, for example, for nasal delivery, may also be prepared in which suitable propellant adjuvants are used. Compositions of the present invention may also be administered as a bolus, electuary, or paste. Compositions for mucosal administration are also encompassed, including oral, nasal, rectal or vaginal administration for the treatment of infections, which affect these areas. Such compositions generally include one or more suitable non-irritating excipients or carriers comprising, for example, cocoa butter, polyethylene glycol, a suppository wax, a salicylate or other suitable carriers. Other adjuvants may also be added to the composition regardless of how it is to be administered, which, for example, may aid to extend the shelf-life thereof.

The combination of β-lactam antibiotic with a non-antibiotic compound as defined herein advantageously provides a synergistic anti-bacterial composition, i.e. a composition that exhibits activity that is greater than the expected additive activity of the β-lactam antibiotic with the non-antibiotic compound. In fact, the β-lactam antibiotic exhibits minimal inhibitory activity against target bacteria, while the non-antibiotic compounds exhibit little or no inhibitory activity against these bacteria. However, in combination, the β-lactam antibiotic and non-antibiotic compound form a synergistic composition that is very effective to inhibit Staphylococcal species, such as MRSA and MSSA.

Thus, in another aspect of the invention, a method of treating a bacterial infection, e.g. Staphylococcal infection, in a mammal is provided. The method includes the step of administering to the mammal a PBP2-binding β-lactam antibiotic and a non-antibiotic compound having the general formula (I) as detailed above. Examples of suitable non-antibiotic compounds include compounds having the general formula (II) and (III). The antibiotic may be administered to the mammal either together with the non-antibiotic compound or the compounds may be separately administered.

The terms “treat”, “treating” and “treatment” are used broadly herein to denote methods that at least reduce one or more adverse affects of a bacterial infection, including those that moderate or reverse the progression of, reduce the severity of, prevent, or cure the infection.

The term “mammal” as it is used herein is meant to encompass humans as well as non-human mammals such as domestic animals (e.g. dogs, cats and horses), livestock (e.g. cattle, pigs, goats, sheep) and wild animals.

The β-lactam antibiotic and non-antibiotic compounds may be administered via any suitable route. The antibiotic and non-antibiotic compounds may be administered together by the same or different route, or concurrently via the same or different routes. As will be appreciated by the skilled artisan, the route and/or mode of administration may vary on a number of factors, including for example, the compounds to be administered and the infection to be treated. Routes of administration include parental, such as intravenous, intramuscular, intradermal, intraperitoneal, subcutaneous, spinal, intraarterial, intrathecal, intracapsular, intraorbital, intracardiac, transtracheal, subcuticular, intraarticular, subcapsular, subarachnoid, intraspinal, epidural and intrasternal injection and infusion. Alternatively, non-parenteral routes may be used, including topical, epidermal or mucosal routes of administration, for example, intranasally, orally, vaginally, rectally, sublingually or topically.

Effective dosage levels of the β-lactam antibiotic and non-antibiotic compounds will vary with factors such as the mammal being treated, the compounds selected for use, and mode of administration. A “therapeutically effective dosage” of each of the β-lactam antibiotic and non-antibiotic compounds is a dosage that is effective to treat an infection by S. aureus. Therapeutically effective dosages of each of the β-lactam antibiotic and non-antibiotic compounds is a dosage of between about 1-500 mg.

Embodiments of the invention are described in the following specific examples which are not to be construed as limiting.

### EXAMPLE 1

### Identification of Antibacterial Effect of Cefuroxime Combinations

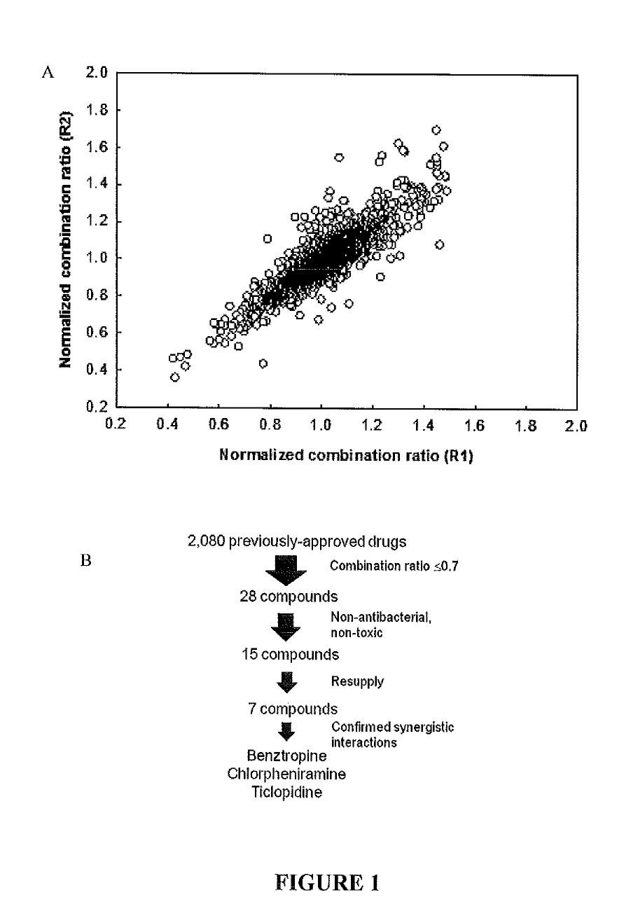

A systematic high-throughput screen of a collection of 2,080 previously approved drugs (PAD library) (10 μM) in combination with the conventional antibiotic, cefuroxime (0.125 μg mL−1), against CA-MRSA USA300 was conducted to look for molecules capable of potentiating its antibacterial activity. CA-MRSA USA300 was screened against the PAD library in the presence of cefuroxime. Screening was carried out in 96-well plates, in duplicate, using Mueller Hinton Broth (MHB) with 2% DMSO and a library compound concentration of 10 μM. The concentration of cefuroxime was 16 μg/ml, a quarter of its MIC value obtained under the same conditions. Background controls (8 wells per plate) contained only media and DMSO and growth controls, also 8 wells per plate, contained media, DMSO and inoculum. Plates were incubated at 37° C. for 20 hours and optical density read at 600 nm using an EnVision plate reader (Perkin Elmer). The percentage growth for each test well was calculated as (OD−mean background)/(mean growth−mean background)*100 and normalized to the percent growth attributed by the PAD alone to obtain a growth ratio such that a ratio of 1.0 is indicative of no difference. A replicate plot is shown (see FIG. 1A) of the screen where R1 and R2 represent the ratio of the percent growth of the various combinations divided by the percent growth of cefuroxime alone of each replicate. Each circle represents this ratio of 1 of 2080 previously approved drugs combined with cefuroxime such that the lower the ratio, the greater the synergistic interaction. Black circles represent active compounds. FIG. 1B is a schematic diagram of the work-flow of the screen conducted from 2,080 compounds to 3 hits. Compounds were eliminated at each stage according to the criteria indicated. Benztropine, chlorpheniramine and ticlopidine were identified as acting synergistically with cefuroxime, inhibiting the growth of CA-MRSA USA 300 by more than 80% .

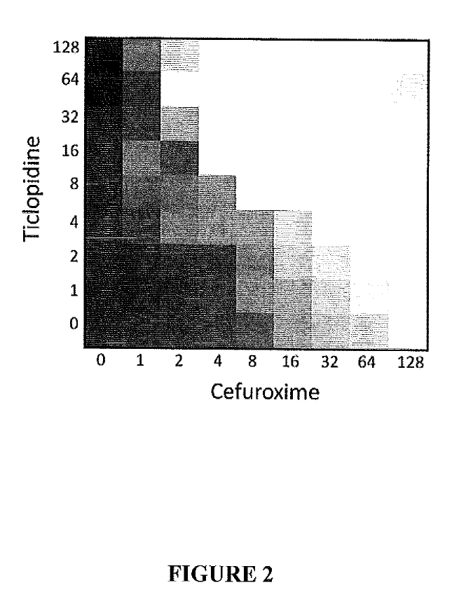

A secondary screen involving detailed checkerboard analyses to characterize the drug-drug interactions confirmed the synergistic interactions between cefuroxime and benztropine, chlorpheniramine and ticlopidine. Briefly, fractional inhibitory concentration (FIC) Index was used to assess interactions between two drugs, whereby an FIC index of 1 indicates no interaction, an FIC index of less than 1 indicates synergy and an FIC index of greater than 2 indicates antagonism. A sample checkerboard analysis of ticlopidine in combination with cefuroxime in CA-MRSA USA 300 shown in FIG. 2. Here a matrix of drug concentrations were tested in combinations at the concentrations shown (μg/mL). Greyscale indicates growth—darker is more growth, lighter is less growth. FIC index is a measure of synergy and is calculated as follows: FIC index=FIC (cefuroxime)+FIC (drug compound). Ticlopidine and cefuroxime show strong synergy (FIC index≦0.12).

### EXAMPLE 2

### Ticlopidine and Cefuroxime Combination Against S. aureus Strains

The combination of ticlopidine and cefuroxime was further tested as above against a variety of S. aureus strains as shown in Table 1 below.

TABLE 1
In vitro interactions between ticlopidine and cefuroxime in S. aureus species

| | MIC | | MIC | | |
|---|---|---|---|---|---|
| | cefuroxime | FIC^{b} | ticlopidine | FIC^{b} | FIC |
| Strain^{a(Ref)} | (μg/mL) | cefuroxime | (μg/mL) | ticlopidine | Index^{c} |
| Newman | 2 | 0.125 | >256 | 0.125 | ≦0.250 |
| HA-MRSA USA600 | ≧1024 | 0.008 | >256 | 0.032 | ≦0.040 |
| HA-MRSA USA100/800/NY | 1024 | 0.125 | >256 | 0.125 | ≦0.250 |
| HA-MRSA | >2048 | 0.250 | >256 | 0.032 | ≦0.282 |
| HA-MRSA USA200/EMRSA16 | 1024 | 0.032 | >256 | 0.063 | ≦0.095 |
| HA-MRSA USA500 | 32 | 1 | >256 | 1 | ≦2 |
| HA-MRSA | >2048 | 0.250 | >256 | 0.016 | ≦0.266 |
| CA-MRSA USA400/MW2 | 256 | 0.063 | >256 | 0.063 | ≦0.125 |
| HA-MRSA EMRSA15 | 512 | 0.063 | >256 | 0.063 | ≦0.125 |
| HA-MRSA | 2048 | 0.125 | >256 | 0.063 | ≦0.188 |
| CA-MRSA USA300 | 512 | 0.032 | >256 | 0.032 | ≦0.063 |
| RN4220 | 0.5 | 1 | >128 | 1 | ≦2 |
| SA178R1 | 0.5 | 1 | >128 | 1 | ≦2 |

^{a}HA, hospital-associated isolate, CA, community-associated isolate
^{b}Fractional Inhibitory Concentration (FIC) = [X]/MICₓ, where [X] is the lowest inhibitory concentration of drug in the presence of the co-drug.
^{c}FIC index = FIC_{cefuroxime} + FIC_{ticlopidine}

The combination exhibited a synergistic effective against a number of the strains tested, and showed the greatest synergy and efficacy against pathogenic S. aureus strains. Notably, the potent synergy was retained in 7 of 10 distinct methicillin-resistant strains from the Canadian MRSA collection (CMRSA), including the USA300 strain, a leading cause of infection and an exceptionally drug-resistant variant. Interestingly, ticlopidine has no antibacterial activity alone against these strains, but in combination with a β-lactam potentiated the activity of the β-lactams by over 32-fold, even in β-lactam-resistant S. aureus.

### EXAMPLE 3

### TiclopidineCombinations Against S. aureus

An extensive analysis of ticlopidine in combination with a wide spectrum of antibiotics, including 13 different β-lactams, against S. aureus (Newman strain) revealed synergistic interactions specifically with the latter class of molecules (Table 2).

TABLE 2

| Target | Antibiotic | FIC index |
|---|---|---|
| Cell wall | Cephalosporin C | ≦0.75 |
| | Cephalexin | ≦1 |
| | Cefuroxime | ≦0.25 |
| | Cefalcor | ≦1 |
| | Cefamandole | ≦0.75 |
| | Cefmetazole | ≦0.75 |
| | Cefoxitin | ≦0.75 |
| | Ceftazidime | ≦1 |
| | Cefsulodin | ≦1 |
| | Ceftoxamine | ≦1 |
| | Methicillin | ≦1 |
| | Piperacillin | ≦0.63 |
| | Methicillin | ≦1 |
| | Ampicillin | ≦1 |
| | Oxacillin | ≦0.63 |
| | Nafcillin | ≦1 |
| | Vancomycin | ≦2 |
| | Bacitran | ≦2 |
| | Daptomycin | ≦2 |
| | D-cycloserine | ≦2 |
| | Fosfomycin | ≦0.5 |
| | Tunicamycin | ≦0.75 |
| | Polymyxin | ≦0.5 |
| Antifolate | Trimethoprim | ≦2 |
| DNA synthesis | Ciprofloxacin | ≦2 |
| | Levofloxacin | ≦2 |
| Ribosome | Tobramycin | ≦2 |
| | Clarithromycin | ≦2 |
| | Erythromycin | ≦2 |

### EXAMPLE 4

### Ticlopidine and Antibiotic Combinations Against CA-MRSA USA 300

Combination studies of ticlopidine with a panel β-lactam of antibiotics against CA-MRSA USA 300 showed similar specificity, resulting in synergistic interactions with 12 of the 20 β-lactams surveyed (Table 3). FIC index=FIC (β-lactam)+FTC (ticlopidine).

TABLE 3

| Antibiotic | FIC index |
|---|---|
| Ampicillin | ≦0.5 |
| Cefaclor | ≦0.5 |
| Cefadroxil | ≦2 |
| Cefamandole | ≦0.63 |
| Cefotaxime | ≦0.5 |
| Cefoxitin | ≦0.5 |
| Cefsulodin | ≦2 |
| Ceftazidime | ≦2 |
| Ceftizoxime | ≦0.5 |
| Cefuroxime | ≦0.13 |
| Cephalexin | ≦0.75 |
| Cephalothin | ≦0.5 |
| Cephradine | ≦2 |
| Cloxacillin | ≦0.5 |
| Meropenem | ≦0.75 |
| Methicillin | ≦0.63 |
| Nafcillin | ≦0.5 |
| Oxacillin | ≦0.5 |
| Penicillin G | ≦0.5 |
| Piperacillin | ≦0.5 |

### EXAMPLE 5

### Thienopyridine and Cefuroxime Combinations Against CA-MRSA USA300

Ticlopidine belongs to a class of compounds known as thienopyridines, a class of antiplatelet drugs known as ADP receptor inhibitors commonly used to treat cardiovascular disease. Like ticlopidine, other thienopyridine analogs, clopidogrel and prasugrel, also show strong synergistic interactions with β-lactams. Specifically, clopidogrel in combination with cefuroxime against CA-MRSA USA300 resulted in an FIC of ≦0.20, while prasugrel resulted in an FTC index of ≦0.50 (Table 4).

TABLE 4

| | MIC | | MIC | | |
|---|---|---|---|---|---|
| | thienopyridine | FIC | Cefuroxime | FIC | FIC |
| Thienopyridine | (μg/mL) | thienopyridine | (μg/mL) | Cefuroxime | Index |
| Ticlopidine | >128 | 0.13 | 256 | 0.03 | ≦0.16 |
| (Ticlid) | | | | | |
| Clopidogrel | >128 | 0.13 | 256 | 0.063 | ≦0.20 |
| (Plavix) | | | | | |
| Prasugrel | >128 | 0.25 | 256 | 0.25 | ≦0.50 |
| (Effient) | | | | | |

### EXAMPLE 6

### Non-Thienopyridines and Cefuroxime Combinations Against MRSA

A series of analogues were synthesized (as shown in Table 5) in which the thienopyridine ring was removed. These were tested as described above and a number of them were found to exhibit synergy with the beta-lactam antibiotic, Cefuroxime, against MRSA.

TABLE 5

| | | | FIC |
|---|---|---|---|
| Compound | | Structure | Index |
| 1 (Ticlopidine) 5-(2-Chloro-benzyl)-4,5,6,7-tetrahydro-thieno[3,2-c]pyridine | | 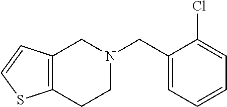 [SMILES: Clc1ccccc1CN1CCc2sccc2C1] | ≦0.125 |
| 2 4,5,6,7-Tetrahydro-thieno[3,2-c]pyridine | | 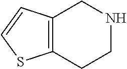 | ≦2 |
| 3 2-(2-Chloro-benzyl)-1,2,3,4-tetrahydro-isoquinoline | | 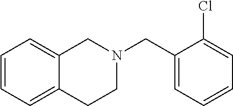 [SMILES: Clc1ccccc1CN1CCc2ccccc2C1] | ≦0.125 |
| 4 2-(3-Chloro-benzyl)-1,2,3,4-tetrahydro-isoquinoline | | 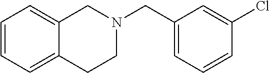 [SMILES: Clc1cccc(CN2CCc3ccccc3C2)c1] | ≦2 |
| 5 2-(4-Chloro-benzyl)-1,2,3,4-tetrahydro-isoquinoline | | 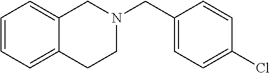 [SMILES: Clc1ccc(CN2CCc3ccccc3C2)cc1] | ≦2 |
| 6 2-Benzyl-1,2,3,4-tetrahydro-isoquinoline | | 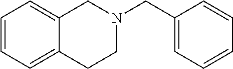 [SMILES: c1ccc(CN2CCc3ccccc3C2)cc1] | ≦0.625 |
| 7 2-Pyridin-4-ylmethyl-1,2,3,4-tetrahydro-isoquinoline | | 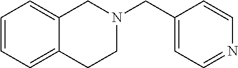 [SMILES: c1ccc2c(c1)CCN(Cc1ccncc1)C2] | ≦2 |
| 8 2-(2-Chloro-benzyl)-isoquinolinium | | 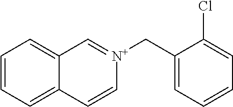 [SMILES: Clc1ccccc1C[n+]1ccc2ccccc2c1] | ≦2 |
| 9 1-(2-Chloro-benzyl)-1,2,3,4-tetrahydro-quinoline | | 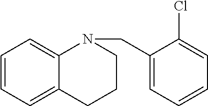 [SMILES: Clc1ccccc1CN1CCCc2ccccc21] | ≦0.187 |
| 10 1-(2-Chloro-benzyl)-4-ethyl-piperazine | | 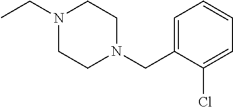 [SMILES: CCN1CCN(Cc2ccccc2Cl)CC1] | ≦2 |
| 11 1-(2-Chloro-benzyl)-pyrrolidine | | 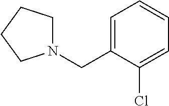 [SMILES: Clc1ccccc1CN1CCCC1] | ≦2 |
| 12 1-(2-Chloro-benzyl)-piperidine | | 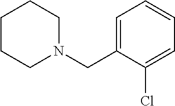 [SMILES: Clc1ccccc1CN1CCCCC1] | ≦2 |
| 13 2-(2-Chloro-benzyl)-decahydro-isoquinoline | | 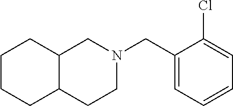 [SMILES: Clc1ccccc1CN1CCC2CCCCC2C1] | ≦2 |
| 14 2-(2-Methyl-benzyl)-1,2,3,4-tetrahydro-isoquinoline | | 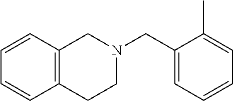 [SMILES: Cc1ccccc1CN1CCc2ccccc2C1] | ≦0.125 |
| 15 2-(2-Fluoro-benzyl)-1,2,3,4-tetrahydro-isoquinoline | | 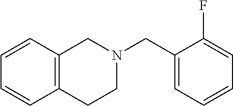 [SMILES: Fc1ccccc1CN1CCc2ccccc2C1] | ≦0.187 |
| 16 2-(2-Nitro-benzyl)-1,2,3,4-tetrahydro-isoquinoline | | 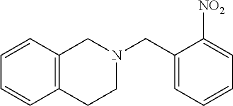 [SMILES: O=[N+]([O-])c1ccccc1CN1CCc2ccccc2C1] | ≦0.187 |
| 17 2-(2-Trifluoromethyl-benzyl)-1,2,3,4-tetrahydro- isoquinoline | | 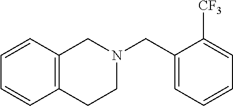 [SMILES: FC(F)(F)c1ccccc1CN1CCc2ccccc2C1] | ≦0.094 |
| 18 (2-Chloro-phenyl)-(3,4-dihydro-1H-isoquinolin-2-yl)-acetic acid methyl ester | | 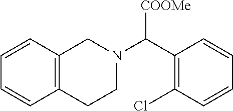 [SMILES: COC(=O)C(c1ccccc1Cl)N1CCc2ccccc2C1] | ≦0.187 |
| 19 (Clopidogrel) | Sigma Clop | 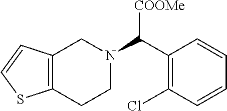 | ≦0.187 |
| 20 | KK20 | 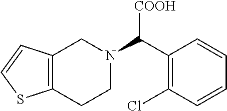 | ≦0.75 |
| 21 | KK21 | 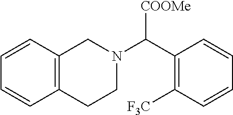 [SMILES: COC(=O)C(c1ccccc1C(F)(F)F)N1CCc2ccccc2C1] | ≦0.125 |
| 22 | KK22 | 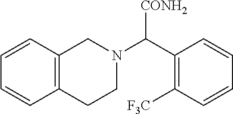 [SMILES: NC(=O)C(c1ccccc1C(F)(F)F)N1CCc2ccccc2C1] | ≦1 |
| 23 | KK23 | 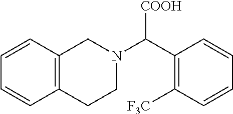 [SMILES: O=C(O)C(c1ccccc1C(F)(F)F)N1CCc2ccccc2C1] | ≦2 |

### EXAMPLE 7

### Novel Non-Thienopyridines and Cefuroxime Combinations

A series of novel analogues were synthesized (as shown in Table 6) which do not include a thienopyridine ring. These were tested in combination with cefuroxime against MRSA and results are shown below (Table 6).

TABLE 6

| | | FIC |
|---|---|---|
| Compound | Structure | Index¹ |
| 1-novel 6-Chloro-2-(2-chloro-benzyl)-1,2,3,4-tetrahydro- isoquinoline | 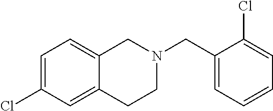 [SMILES: Clc1ccc2c(c1)CCN(Cc1ccccc1Cl)C2] | ≦2 |
| 2-novel 2-(2-Chloro-benzyl)-1,2,3,4-tetrahydro-isoquinolin-7-ol | 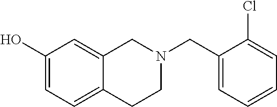 [SMILES: Oc1ccc2c(c1)CN(Cc1ccccc1Cl)CC2] | ≦2 |
| 3-novel 2-Naphthalen-2-ylmethyl-1,2,3,4-tetrahydro-isoquinoline | 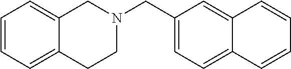 [SMILES: c1ccc2c(c1)CCN(Cc1ccc3ccccc3c1)C2] | ≦0.312 |
| 4-novel (2-Chloro-phenyl)-(3,4-dihydro-1H-isoquinolin-2-yl)-acetic acid | 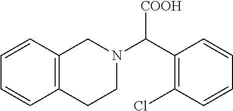 [SMILES: O=C(O)C(c1ccccc1Cl)N1CCc2ccccc2C1] | ≦2 |

We claim:

1. A composition comprising a combination of
i) a β-lactam antibiotic that has a binding affinity for bacterial penicillin-binding protein 2; and
ii) a non-antibiotic compound having the following general formula (I): 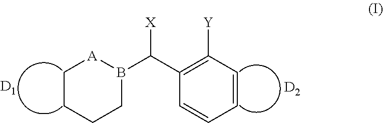
wherein:
A may be C or N, and B may be C or B may be N when A is C;
X is selected from the group consisting of H, OH, CH₂OH, CH₂F, CHF₂, CF₃, C₁-C₆ alkyl, —COOH, —COR, —COOR, NO₂, NH₃, NH₂R, NHR₂, wherein R is selected from cyclic, linear or branched C₁-C₆ alkyl and may be the same or different in NHR₂;
Y is selected from the group consisting of H, OH, halogen (e.g. Br, Cl, F and I), C₁-C₆ alkyl, NO₂, NH₃, NH₂R¹, NH(R¹)₂, CF₃, CH₂OH, CH₂F, CHF₂, wherein R¹ is selected from halogen and linear or branched C₁-C₆ alkyl and may be the same or different in NH(R¹)₂;
D₁ may be phenyl, substituted or unsubstituted thiophene, substituted or unsubstituted furan or substituted or unsubstituted pyrrole, wherein the substituents are selected from OH, halogen (e.g. Br, Cl, F and I), cyclic, linear or branched C₁-C₆ alkyl and OR², wherein R² is selected from C₁-C₆ alkyl and —COR; and
D₂ is either phenyl or nil.

2. The composition of claim 1, wherein the β-lactam antibiotic is selected from the group consisting of Cephalexin Cefuroxime, Cefamandole, Cefalcor, Cefoxitin, Ceftazidime, Ampicillin, Methicillin, Nafcillin, Oxacillin, Penicillin G and Piperacillin.

3. The composition of claim 1, wherein the non-antibiotic compound is a thienopyridine compound having the general formula (II): 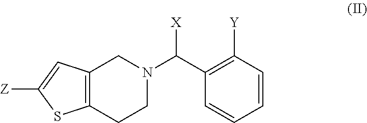
wherein:
X and Y are as defined above; and
Z is selected from H, OH, cyclic, linear or branched C₁-C₆ alkyl and OR², wherein R² is as defined in claim 1.

4. The composition of claim 3, wherein the non-antibiotic compound is selected from the group consisting of ticlopidine, clopidogrel and prasugrel.

5. The composition of claim 1, wherein the non-antibiotic compound is a non-thienopyridine compound having the general formula (III): 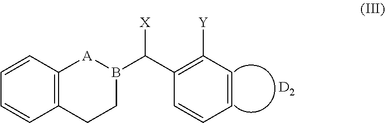
wherein:
A, B, X, Y and D₂ are as defined claim 1.

6. The composition of claim 5, wherein the non-antibiotic compound is selected from the group consisting of 2-(2-Chloro-benzyl)-1,2,3,4-tetrahydro-isoquinoline, 2-Benzyl-1,2,3,4-tetrahydro-isoquinoline, 1-(2-Chloro-benzyl)-1,2,3,4-tetrahydro-quinoline 2-(2-Methyl-benzyl)-1,2,3,4-tetrahydro-isoquinoline, 2-(2-Fluoro-benzyl)-1,2,3,4-tetrahydro-isoquinoline, 2-(2-Nitro-benzyl)-1,2,3,4-tetrahydro-isoquinoline, 2-(2-Trifluoromethyl-benzyl)-1,2,3,4-tetrahydro-isoquinoline, (2-Chloro-phenyl)-(3,4-dihydro-1H-isoquinolin-2-yl)-acetic acid methyl ester and 2-Naphthalen-2-ylmethyl-1,2,3,4-tetrahydro-isoquinoline.

7. The composition of claim 5, wherein Y is selected from the group consisting of H, OH, halogen, C₁-C₆ alkyl, NO₂, NH₃, CF₃, CH₂OH, CH₂F and CHF₂, and X is selected from the group consisting of H, C₁-C₆ alkyl, —COR and —COOR, wherein R is selected from cyclic, linear or branched C₁-C₆ alkyl.

8. A method of treating a bacterial infection in a mammal comprising the step of administering to the mammal an effective amount of a β-lactam antibiotic that has a binding affinity for bacterial penicillin-binding protein 2, and a non-antibiotic compound a non-antibiotic compound having the following general formula (I): 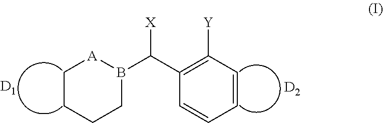
wherein:
A may be C or N, and B may be C or B may be N when A is C;
X is selected from the group consisting of H, OH, CH₂OH, CH₂F, CHF₂, CF₃, C₁-C₆ alkyl, —COOH, —COR, —COOR, NO₂, NH₃, NH₂R, NHR₂, wherein R is selected from cyclic, linear or branched C₁-C₆ alkyl and may be the same or different in NHR₂;
Y is selected from the group consisting of H, OH, halogen (e.g. Br, Cl, F and I), C₁-C₆ alkyl, NO₂, NH₃, NH₂R, NH(R¹)₂, CF₃, CH₂OH, CH₂F, CHF₂, wherein R¹ is selected from halogen and linear or branched C₁-C₆ alkyl and may be the same or different in NH(R¹)₂;
D₁ may be phenyl, substituted or unsubstituted thiophene, substituted or unsubstituted furan or substituted or unsubstituted pyrrole, wherein the substituents are selected from OH, halogen (e.g. Br, Cl, F and I), cyclic, linear or branched C₁-C₆ alkyl and OR², wherein R² is selected from C₁-C₆ alkyl and —COR; and
D₂ is either phenyl or nil.

9. The method of claim 8, wherein the β-lactam antibiotic is selected from the group consisting of Cephalexin Cefuroxime, Cefamandole, Cefalcor, Cefoxitin, Ceftazidime, Ampicillin, Methicillin, Nafcillin, Oxacillin, Penicillin G and Piperacillin.

10. The method of claim 8, wherein the non-antibiotic compound is a thienopyridine compound having the general formula (II): 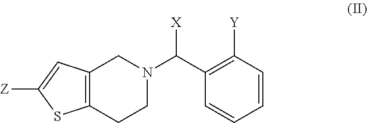
wherein:
X and Y are as defined in claim 8; and
Z is selected from H, OH, cyclic or linear C₁-C₆ alkyl and OR², wherein R² is as defined in claim 8.

11. The method of claim 10, wherein the non-antibiotic compound is selected from the group consisting of ticlopidine, clopidogrel and prasugrel.

12. The method of claim 8, wherein the non-antibiotic compound is a non-thienopyridine compound having the general formula (III): 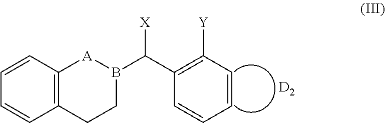
wherein:
A, B, X, Y and D₂ are as defined claim 8.

13. The method of claim 12, wherein the non-antibiotic compound is selected from the group consisting of 2-(2-Chloro-benzyl)-1,2,3,4-tetrahydro-isoquinoline, 2-Benzyl-1,2,3,4-tetrahydro-isoquinoline, 1-(2-Chloro-benzyl)-1,2,3,4-tetrahydro-quinoline 2-(2-Methyl-benzyl)-1,2,3,4-tetrahydro-isoquinoline, 2-(2-Fluoro-benzyl)-1,2,3,4-tetrahydro-isoquinoline, 2-(2-Nitro-benzyl)-1,2,3,4-tetrahydro-isoquinoline, 2-(2-Trifluoromethyl-benzyl)-1,2,3,4-tetrahydro-isoquinoline, (2-Chloro-phenyl)-(3,4-dihydro-1H-isoquinolin-2-yl)-acetic acid methyl ester and 2-Naphthalen-2-ylmethyl-1,2,3,4-tetrahydro-isoquinoline.

14. The method of claim 12, wherein A is C and B is N.

15. The method of claim 12, wherein Y is selected from the group consisting of H, OH, halogen, C₁-C₆ alkyl, NO₂, NH₃, CF₃, CH₂OH, CH₂F and CHF₂.

16. The method of claim 12, wherein X is selected from the group consisting of H, C₁-C₆ alkyl, —COR and —COOR, wherein R is selected from cyclic, linear or branched C₁-C₆ alkyl.

17. A compound having the general formula (III): 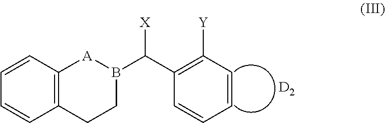
wherein:
A may be C or N, and B may be C or B may be N when A is C;
X is —COOR, wherein R is selected from cyclic, linear or branched C₁-C₆; Y is CF₃ or H, and D₂ is phenyl or nil.

18. The compound of claim 17, wherein A is C, B is H, and X is COOMe or COONH₂.

19. The compound of claim 17 which is Naphthalen-2-ylmethyl-1,2,3,4-tetrahydro-isoquinoline.
